(19) United States
(12) Patent Application Publication
(10) Pub. No.: US 2014/0378809 A1
(43) Pub. Date: Dec. 25, 2014

(54) SYSTEMS AND METHODS FOR EXTRACTING PHYSIOLOGICAL CHARACTERISTICS USING FREQUENCY HARMONICS

(71) Applicants: Mary Ann WEITNAUER, Atlanta, GA (US); Van NGUYEN, Atlanta, GA (US); Abdul Qadir JAVAID, Smyrna, GA (US)

(72) Inventors: Mary Ann WEITNAUER, Atlanta, GA (US); Van NGUYEN, Atlanta, GA (US); Abdul Qadir JAVAID, Smyrna, GA (US)

(21) Appl. No.: 14/247,070

(22) Filed: Apr. 7, 2014

Related U.S. Application Data
(60) Provisional application No. 61/837,547, filed on Jun. 20, 2013.
(60) Provisional application No. 61/954,533, filed on Mar. 17, 2014.

(51) Int. Cl. A61B 5/05; A61B 5/024

(52) U.S. Cl. CPC A61B 5/05; A61B 5/02444
USPC 600/407

(57) ABSTRACT

Methods, systems and computer program products are provided estimating a physiological characteristic of a subject based on a reflected signal received by a UWB radar system. The method comprises: processing a signal received from the UWB radar system to obtain a frequency domain representation of the signal; discerning, from the frequency domain representation, a plurality of elements considered to belong to a physiological characteristic harmonic set {PC₀, PC₁, PC₂, PC₃ . . . }, the physiological characteristic harmonic set comprising a fundamental frequency of the physiological characteristics and harmonic frequencies of the physiological characteristic; and estimating the physiological characteristic based on the discerned elements of the physiological characteristic harmonic set.

### RELATED APPLICATIONS

This application claims the benefit of the priority of U.S. application No. 61/837,547 filed 20 Jun. 2013 and U.S. application No. 61/954,533 filed 17 Mar. 2014, both of which are hereby incorporated herein by reference.

### TECHNICAL FIELD

The subject matter of this disclosure relates to sensing and/or monitoring life characteristics (e.g. physiological characteristics) of human or other animal subjects. Particular aspects of the invention provide systems and methods for using Impulse-Radio Ultra Wideband (IR-UWB or, for brevity, UWB) radar systems to sense and/or monitor such physiological characteristics.

### BACKGROUND

There is a general desire to use IR-UWB radar systems to sense and/or monitor physiological characteristics of human and/or other animal subjects. One non-limiting reason that IR-UWB is desirable because it can be configured so as not to interfere with (or be interfered by) other wireless communication equipment. Also, IR-UWB radar can be used without contacting the subject and can be used at relatively low power, suitable for continuous monitoring.

Prior art spectral analysis techniques for sensing vital signs using radar based systems involve detecting the fundamental peaks of the heart rate and respiration rate. A difficulty with this approach, particularly for, but not limited to, detecting the heart rate, is that the heart rate fundamental can be interfered with or otherwise obscured by respiration harmonics and/or intermodulation products between the heart rate and the respiration rate. In addition, subject movement can impact the accuracy and/or efficacy of techniques based on detecting the fundamental frequency of the heart rate or respiration rate.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides a method for estimating a physiological characteristic of a subject based on a reflected signal received by a UWB radar system. The method comprises: processing a signal received from the UWB radar system to obtain a frequency domain representation of the signal; discerning, from the frequency domain representation, a plurality of elements considered to belong to a physiological characteristic harmonic set {PC0, PC1, PC2, PC3 . . . }, the physiological characteristic harmonic set comprising a fundamental frequency of the physiological characteristics and harmonic frequencies of the physiological characteristic; estimating the physiological characteristic based on the discerned elements of the physiological characteristic harmonic set.

Another aspect of the invention provides a system for estimating a physiological characteristic of a subject. The system comprises: a UWB radar system for directing UWB pulses toward the subject, receiving reflected pulses and generating a signal based on the reflected pulses; and a processor connected to receive the signal from the UWB radar system. The processor (which may comprise one or more processors) is configured to: process the signal to obtain a frequency domain representation of the signal; discern, from the frequency domain representation, a plurality of elements considered to belong to a physiological characteristic harmonic set {PC0, PC1, PC2, PC3 . . . }, the physiological characteristic harmonic set comprising a fundamental frequency of the physiological characteristics and harmonic frequencies of the physiological characteristic; and estimate the physiological characteristic based on the discerned elements of the physiological characteristic harmonic set.

Another aspect of the invention provides a computer program product program product comprising a non-transitory computer-readable medium having executable code configured to cause a processor executing the code to perform a method for estimating a physiological characteristic of a subject based on a reflected signal received by a UWB radar system, the method comprising: processing a signal received from the UWB radar system to obtain a frequency domain representation of the signal; discerning, from the frequency domain representation, a plurality of elements considered to belong to a physiological characteristic harmonic set {PC0, PC1, PC2, PC3 . . . }, the physiological characteristic harmonic set comprising a fundamental frequency of the physiological characteristics and harmonic frequencies of the physiological characteristic; and estimating the physiological characteristic based on the discerned elements of the physiological characteristic harmonic set.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1A is a block diagram of an ultra-wideband (UWB) sensing system for estimating one or more physiological characteristics of a human or other animal according to a particular exemplary embodiment. FIG. 1B is a schematic view showing the FIG. 1A UWB sensing system estimating one or more physiological characteristics of a human subject through a mattress by way of UWB pulses. FIG. 1C is a schematic depiction of a monitoring system according to a particular exemplary embodiment which may incorporate one or more of the FIG. 1A UWB sensing systems.

FIG. 2 is a block diagram representation of a method for estimating a physiological characteristic of a human or other animal according to a particular embodiment which may be performed by the FIG. 1A UWB sensing system.

FIG. 3 is a schematic depiction of filter that may be applied to data received by the radar system of the FIG. 1A sensing system for separating the respiration fundamental from the heart rate fundamental of a subject and for removing DC components.

FIG. 4 shows an example of the type of frequency domain data that could be obtained as a part of the FIG. 2 physiological characteristic estimation method according to some embodiments.

FIG. 5A is a block diagram representation of a method for obtaining frequency domain data that may be used in the FIG. 2 physiological characteristic estimation method according to some embodiments. FIG. 5B is a schematic depiction of a technique for parsing time domain measurement data into temporal windows, transforming the data from the time domain windows into the frequency domain and averaging the windows of the corresponding frequency domain data which may be used in the method of FIG. 5A according to a particular embodiment.

FIG. 6A is a block diagram representation of a method for discerning harmonic(s) (e.g. elements of the heart rate harmonic set {HR₀, HR₁, HR₂, HR₃, . . . }) which may be used in the FIG. 2 physiological characteristic estimation method according to some embodiments and one particular optional technique which may be used to search for paths according to a particular embodiment. FIG. 6B is a schematic depiction showing a set of peaks which may be the result of a peak location process used in the method of FIG. 6A according to a particular embodiment. FIG. 6C is a schematic depiction showing a set of paths which may be the result of the path search process used in the method of FIG. 6A according to a particular embodiment.

FIG. 7A is a block diagram representation of a method for using discerned harmonic(s) to estimate a physiological characteristic (e.g. heart rate) which may be used in the FIG. 2 physiological characteristic estimation method according to some embodiments.

FIG. 8A is a block diagram representation of a method for discerning harmonic(s) (e.g. elements of the heart rate harmonic set {HR₀, HR₁, HR₂, HR₃, . . . }) which may be used in the FIG. 2 physiological characteristic estimation method according to some embodiments. FIGS. 8B and 8C show experimental data for a pair of successive iterations of the method of FIG. 2 and how the method of FIG. 8A can be used when paths are not located in the original frequency domain data.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

Aspects of the invention provide systems and methods for estimating a physiological characteristic of a human or other animal. Time domain measurement data may be obtained using a UWB radar system. The time domain measurement data may be converted to the frequency domain to obtain a frequency domain representation of the measurement data (or, for brevity, frequency domain data). The system may comprise a processor configured to (or the method may comprise) processing the frequency domain data to discern one or more features of the frequency domain data considered to be representative of one or more harmonic(s) of a physiological characteristic sought to be estimated. In some cases, the processor may be configured to (or the method may comprise) discerning a fundamental frequency of the physiological characteristic sought to be estimated. The controller may be configured to (or the method may comprise) using the one or more harmonics (and possibly the fundamental frequency, where discernable) to estimate the physiological characteristic. In some embodiments, the physiological characteristic comprises a heart rate of a human or animal subject. In some embodiments, the physiological characteristic comprises a respiration rate of a human or animal subject.

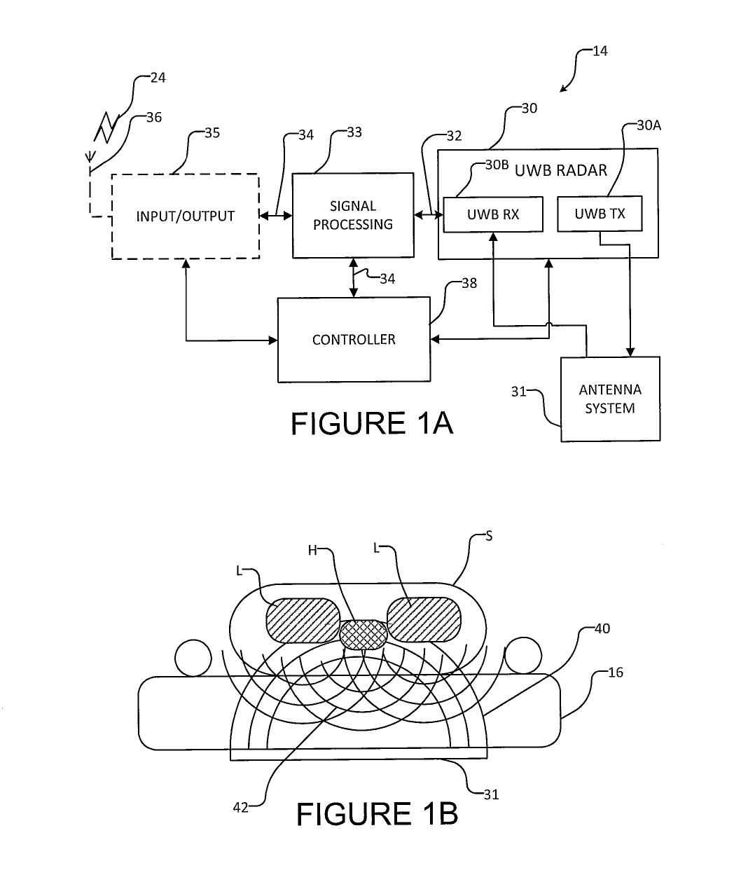

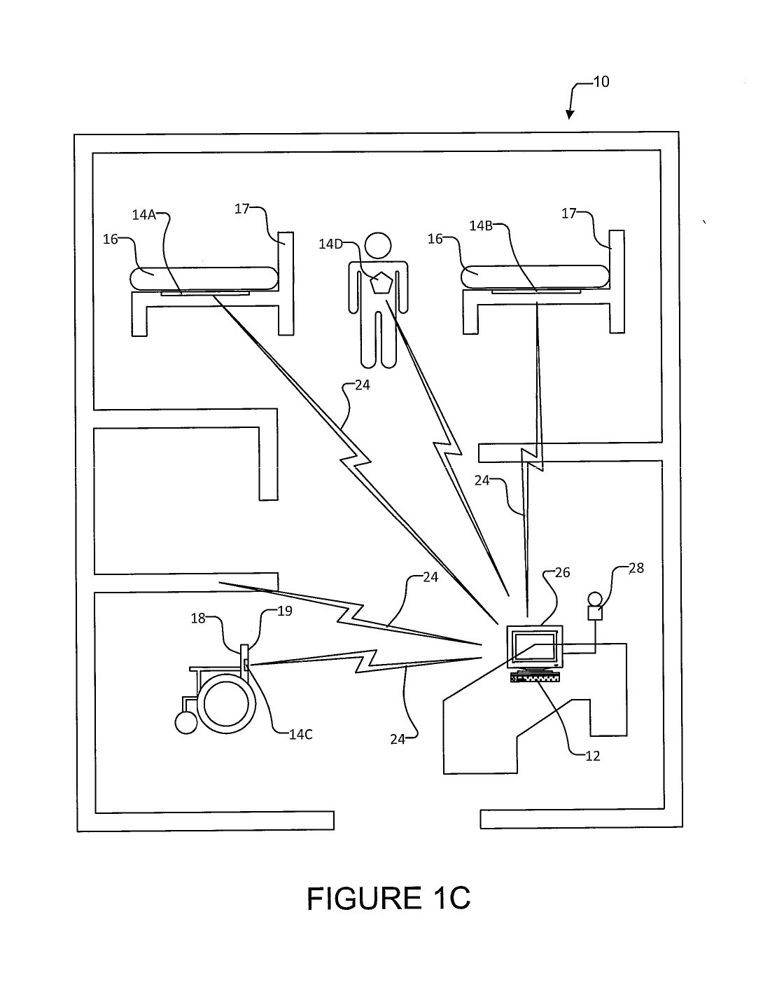

FIG. 1A is a block diagram of an ultra-wideband (UWB) sensing system 14 for estimating one or more physiological characteristics of a subject (not shown) according to a particular exemplary embodiment. Sensing system 14 may estimate one or more physiological characteristics of a human or animal subject. For brevity but without loss of generality, the remainder of this description, except where otherwise noted or where otherwise discernable from the context, assumes that sensing system 14 is used on a human subject. Sensing system 14 may be used to detect a number of physiological characteristics of the subject, including without limitation, heart rate (HR) and/or respiration rate (RR). For brevity but without loss of generality, the remainder of this description, except where otherwise noted or where otherwise discernable from the context, assumes that sensing system is used to detect a heart rate of the subject.

In the illustrated embodiment, the operation of sensing system 14 is controlled by a suitably programmed controller 38. Controller 38 (and/or components thereof) may comprise hardware, software, firmware or any combination thereof. For example, controller 38 may be implemented on a programmed computer system comprising one or more processors, user input apparatus, displays and/or the like. Controller 38 may be implemented as an embedded controller with a suitable user interface comprising one or more processors, user input apparatus, displays and/or the like. Processors may comprise microprocessors, digital signal processors, graphics processors, field programmable gate arrays, and/or the like. Components of controller 38 may be combined or subdivided, and components of controller 38 may comprise sub-components shared with other components of controller 38. Components of controller 38, may be physically remote from one another. In some embodiments, controller 38 is not necessary and the operation of sensing system 14 may be controlled from a central monitoring station or the like which is remote from sensing system 14.

Sensing system 14 comprises a UWB radar system 30 connected to an antenna system 31. UWB radar system 30 comprises a UWB transmitter 30A, and a UWB receiver 30B. UWB radar system 30 generates UWB pulses (or impulses) that are transmitted by antenna system 31 into a space where the subject may be located. This space may be referred to as the “sensing volume” of sensing system 14, since UWB radar system 30 can be used to estimate the physiological characteristics of a person in the sensing volume. If a person is present in the sensing volume, then the pulses transmitted by UWB transmitter 30A are reflected at interfaces within the person's body (e.g. the surfaces of the lungs and heart) and the corresponding reflected pulses may be received at antenna 31 and detected by UWB receiver 30B.

In some embodiments, the UWB pulses used by UWB radar system 30 are in the C-band (e.g. in a range of 3 to 5 GHz in some embodiments). In some embodiments, the width of transmitted UWB pulses may be in a range of 1-20 ns, for example. UWB pulses are transmitted by UWB radar system 30 at a suitable pulse rate. This pulse rate may be set to a value low enough that the average power emitted by radar system 30 is low enough to satisfy applicable regulatory requirements. For example, the pulse repetition interval (PRI) is in the range of 0.5 us to 1 μs in some embodiments. The time-averaged transmitted output power may be relatively small. For example, in some embodiments, the maximum effective isotropic radiation power (EIRP) may be −41.3 dBm/MHz or less.

UWB radar system 30 generates a receiver output signal 32 based at least in part on reflected UWB pulses received by UWB radar receiver 30B. UWB radar system 30 may comprise signal conditioning components which are not expressly shown in FIG. 1A for conditioning the signal received by UWB radar receiver 30B and thereby generating receiver output signal 32. Such signal conditioning components (e.g. circuitry, hardware and/or software) are well understood by those skilled in the art and are not described in detail here. By way of non-limiting example, such signal conditioning components may comprise amplifiers, filters, oscillators, mixers, gating circuits, analog to digital converters, sampling circuits and/or the like.

UWB radar system outputs a receiver output signal 32 which is passed to a signal processing system 33. Signal processing system 33 may comprise one or more digital signal processing (DSP) components configured with suitable DSP software for processing receiver output signal 32. In some embodiments, methods for estimating one or more physiological characteristics described herein may be implemented by signal processing system 33 and/or by signal processing system 33 under the control of controller 38 and may comprise using receiver output signal 32 to estimate one or more physiological characteristics of a subject.

In the illustrated embodiment, signal processing system 33 outputs a signal 34 which is provided (either directly, or via controller 38) to optional input/output system 35. Output signal 34 may comprise information that is representative of the estimated physiological parameters determined by sensing system 14. In some embodiments, input/output system 35 may comprise a suitable user interface for providing output to users (e.g. visual output on a display and/or the like and/or audio output via speakers and/or the like) and/or for receiving input from users (e.g. via a keyboard, a pointing device, a touch screen interface and/or the like). User interfaces are well known and are not described further here. In some embodiments, for example where sensing system 14 is part of a monitoring system 10 (see FIG. 1B), input/output system 35 may comprise a communication device (not expressly shown), such as a wireless communication device, which communicates a signal 24 comprising the estimated physiological characteristic to a remote monitoring station 12, by way of optional antenna 36.

FIG. 1B illustrates schematically how antenna system 31 of sensing system 14 located under a mattress 16 can emit UWB pulses 40, which pass into a subject S and are reflected at various surfaces, including surfaces of the subject's lungs L and heart H, to form reflected pulses 42 that are in turn detected by antenna system 31. By using UWB radar system 30 in a back-scattering remote sensing mode, sensing system 14 can estimate physiological characteristics (e.g. heart and respiration rates) of a subject S without electrodes or other devices being attached to the body of subject S.

Antenna system 31 may comprise an array of transmit antennas (not expressly shown) and an array of receive antennas (not expressly shown). The transmit and receive antennas may be distributed over an area broad enough to be able to transmit UWB pulses 40 and detect reflected UWB pulses 42 from subject S in any reasonable position and posture on mattress 16. The transmit antennas may be low-gain antennas (relative to the receive antennas). The use of low-gain transmit antennas permits transmission of UWB signals having higher average amplitudes without causing EIRP to exceed thresholds that maybe specified by applicable regulations. Further, low-gain antennas generally have broad radiation patterns the radiation is distributed into a broad angular sensing volume. The receive antennas of antenna system 31 may comprise higher-gain antennas (than the transmit antennas) to provide better signal-to-noise ratios (SNR) for received signals ascertained from reflected pulses 42.

While not necessary, sensing system 14 may be part of a monitoring system (e.g. a patient monitoring system). FIG. 1C is a schematic depiction of a monitoring system 10 which may comprises one or more sensing systems 14 according to an example embodiment. System 10 may be used, for example, in a hospital ward, a long term care facility, a nursery, or the like. System 10 comprises a monitoring station 12 and one or more sensing systems 14 (which may be referred to as sensing units 14 when implemented as part of a monitoring system 10). In the illustrated embodiment, sensing units 14 include sensing units 14A and 14B that are located under the mattresses 16 of beds 17, a sensing unit 14C that is built into (or mounted on) the backrest 18 of a wheelchair 19 and a sensing unit 14D that is strapped to a person's chest (as shown) or wrist (not shown).

Monitoring station 12 may receive signals from additional sensors (not shown) in addition to sensing units 14. For example, monitoring station 12 may receive signals from door switches, proximity sensors, other patient monitoring devices, such as, by way of non-limiting example, EEG machines, blood oxygen sensors and/or the like.

Each sensing unit 14 is in communication with monitoring station 12. The communication is preferably wireless communication, although some embodiments may incorporate wired communication. As described below, sensing units 14A, 14B, 14C and 14D may estimate one or more physiological characteristics (e.g. vital signs, such as the heart rate or respiration rate) of a subject at the location of the sensing unit 14. Data signals 24 that contain one or more estimated physiological characteristics from sensing units 14 may be transmitted to monitoring station 12, where they may be analyzed or further processed to monitor other phenomena, such as, by way of non-limiting example: whether the subject's heart rate or breathing rate have stopped or exhibit abnormalities or exhibit sudden changes and/or the like.

Monitoring station 12 of the illustrated embodiment comprises a suitable user interface 26 and a wired or wireless communication module (not expressly shown). This communication module may receive data signals 24 from sensing units 14. Via its user interface 26, monitoring station 12 displays information regarding the various subjects being monitored. User interface 26 may also receive input from authorized users. In some embodiments, user interface 26 may comprise an alarm 28 (e.g. an audible alarm 28). User interfaces are well known and are not described further here. User interface 26 may permit personnel to observe the physiological characteristic(s) of subjects S and alarm 28 may warn such personnel when there is a condition or combination of conditions for which monitoring station 12 may be configured to provide an alarm.

Other suitable sensing systems, antenna systems and monitoring systems which may be used to determine one or more physiological characteristics in accordance with the techniques disclosed herein are described in PCT publication No. WO/2007/101343 which is hereby incorporated herein by reference. Other suitable sensing, antenna and monitoring systems which may be used to determine one or more physiological characteristics in accordance with the techniques disclosed herein are manufactured by Sensiotec Inc. of Atlanta, Ga. under the trademark The Virtual Medical Assistant™.

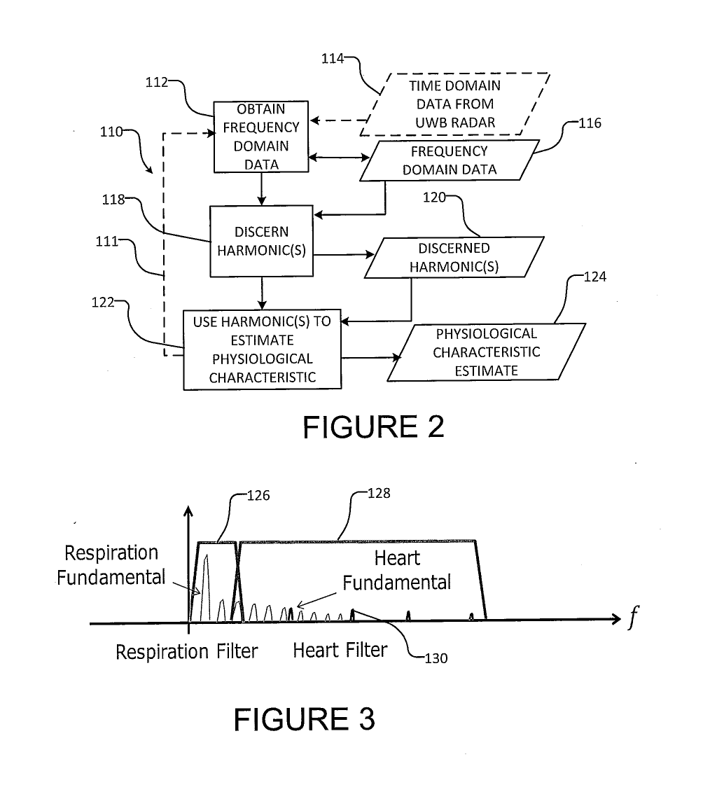

FIG. 2 is a block diagram representation of a method 110 for estimating a physiological characteristic of a subject animal according to a particular embodiment. Method 110 may be performed by UWB sensing system 14 (e.g. by digital signal processing system 33 or digital signal processing system 33 in combination with controller 28). In some embodiments, method 110 may be performed in part by UWB radar system 30 under the control of signal processing system 33 and/or controller 28.

Method 110 starts in block 112 which comprises obtaining frequency domain data 116. In the illustrated embodiment, block 112 comprises processing time domain measurement data 114 output from a UWB radar system (e.g. UWB radar system 30 of sensing system 14 of FIG. 1A) to obtain frequency domain data 116. When UWB transmitter 30A transmits pulses (e.g. pulses 40 shown in FIG. 1B) with a period T, these pulses are reflected (e.g. reflected pulses 42) from the various surfaces of the body of the subject and have a time varying delay which varies with movement of the various surfaces of the body and may be modelled by the equation:

r(t)=Σ_{n=-∞}^{+∞}αₕp(t−nT−τₕ(t))+αₗp(t−nT−τₗ(t))+αₛp(t−nT−τₛ(t)) (1)

where the first term in the sum is representative of reflections from the heart of the subject, the second term in the sum is representative of reflections from the lungs of the subject and the third term in the sum is representative of reflections from skin of the subject. In equation (1): p(•) represents a particular pulse, T is the pulse repetition period, αₕ, αₗ, αₛ are amplitude of the reflected pulses coming from heart, lung and skin interface while τₕ, τₗ and τₛ is the time delay of the reflected pulses coming from heart, lungs and skin. It should be noted that the time delay of the reflected pulses from the skin τₛ(t)=βₕτₕ(t)+βₗτₗ(t) carries information about the delay caused by the lungs and the heart. When reflected pulses 42 are received at radar system 30, the radar received signal spectrum may contain spectral components centered at multiples of the respiration rate (RR), multiples of the heart rate (HR), and their intermodulation products: mRR+lHR, where m and l are integers.

Time domain measurement data 114 may comprise a version of the radar-received signal received by UWB radar receiver 30B which may be down-converted to baseband. As is known in the art, such down-conversion may be performed by suitable components (not expressly shown) of UWB radar receiver 30B. In some embodiments, after down-conversion, time domain measurement data 114 may be pre-filtered by analog filters (not expressly shown) prior to being received in block 112. Such analog pre-filters may be a part of UWB radar receiver 30B and may comprise band-pass filters which attempt to block DC and to separate the fundamental frequency of the respiration rate from the fundamental frequency of the heart rate. This pass-band filtering process is shown schematically in FIG. 3, which depicts an exemplary spectrum of a radar received signal, a respiration filter 126 and a heart filter 128. In typical circumstances, the fundamental frequency of the respiration rate is lower and stronger than the fundamental frequency of the heart rate. Accordingly, as shown in FIG. 3, the pass bands of respiration filter 126 and heart filter 128, which may be slightly overlapping, may be designed to separate the respiration fundamental from the heart fundamental and may also be designed to block DC.

Where the physiological characteristic of interest (e.g. the physiological characteristic estimated by method 110) is the heart rate of the subject, then the signal of interest may be the band-pass filtered signal 130 which is passed by the heart filter 128. Accordingly, in some embodiments where the physiological characteristic of interest is the heart rate, time domain measurement data 114 received in block 112 of FIG. 2 may comprise this band-pass filtered signal 130 shown in FIG. 3 (or a digitally sampled version thereof). It will be appreciated from FIG. 3 that while filters 126, 128 can separate the fundamental frequencies of the heart rate and the respiration rate, the harmonics of both the respiration rate and the heart rate are present in the filter band of heart filter 128 and in the corresponding band-pass filtered signal 130.

In some embodiments, time domain measurement data 114 received from UWB radar system 30 may be sampled and digitized prior to being received in block 112 (e.g. by suitable digital sampling components (not expressly shown) which may form part of UWB receiver 30B). Such digital sampling components are well known in the art. Unlike some prior art techniques which involve sampling rates of tens of GHz, the sampling of time domain measurement data 114 may be performed at a relatively low rate. In one particular embodiment, the sampling rate of time domain measurement data 114 is around 128 Hz. In some embodiments, this sampling rate is less than or equal to 256 Hz. In some embodiments, this sampling rate is less than or equal 1024 Hz. In some embodiments, time domain measurement data 114 received from UWB radar system 30 may still be in the analog domain and may be sampled and digitized as a part of block 112. The block 112 sampling rates may be comparable to those where the sampling and digitizing is performed by UWB receiver 30B

In the illustrated embodiment, block 112 comprises converting time domain measurement data 114 into frequency domain data 116. Frequency domain data 116 may comprise an estimate of the power spectral density, amplitude spectral density or of the reflected pulses received by UWB receiver 30B (and the corresponding time domain measurement data 114). In general, any suitable technique may be used to convert or transform time domain measurement data 114 to the frequency domain to obtain frequency domain data 116. The particular technique used to generate frequency domain data 116 may depend on the nature of time domain measurement data 114. In some embodiments, block 112 may involve application of a digital DC removal filter and/or a Hamming window filter to time domain measurement data 114 prior to transforming time domain measurement data 114 to the frequency domain. In particular non-limiting example embodiments, block 112 comprises applying a discrete Fourier transform (DFT) to a corresponding finite duration window of digitally sampled time domain measurement data 114 (optionally after DC removal and Hamming windowing) and then squaring the magnitude of the resultant frequency domain signal to obtain frequency domain data 116 which comprises a magnitude squared DFT of MSDFT. Any suitable technique may be used to compute the DFT, such as, by way of non-limiting example, any suitable FFT technique. In other embodiments, other suitable spectral transform techniques or other spectral density estimation techniques could be used in the place of the MSDFT to obtain frequency domain data 116. Such spectral transform techniques include, by way of non-limiting example, any suitable Fourier Transform technique, any suitable time-frequency transform technique, any suitable transform involving a weighted sum of periodic functions, and/or the like. In some embodiments, it is not necessary to square the amplitude of the transformed data and method 110 may involve using the absolute value of the magnitudes of the transformed data frequency domain data 116.

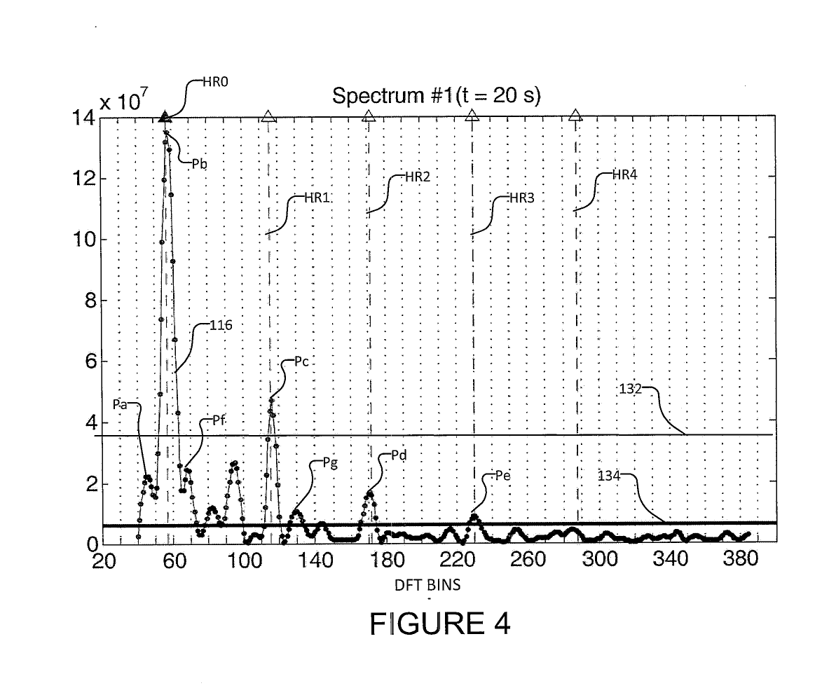

FIG. 4 shows an example of the type of frequency domain data 116 that could be obtained from block 112 according to some embodiments. The data shown in the particular example of FIG. 4 may be obtained in block 112 by application of a DFT to a 20 s window of digitally sampled time domain data and then squaring the result to obtain a MSDFT, although, as discussed above, data similar to that shown in FIG. 4 could be obtained using other techniques. The units shown on the x-axis of the FIG. 4 plot are DFT bins. Those skilled the art will appreciate that there is a relationship between DFT bins shown in FIG. 4 and the frequency of the digitally sampled time domain data which depends on the time domain sampling rate and the length of the window over which the DFT is obtained. In the illustrated data shown in FIG. 4, the heart rate in beats per minute (bpm) may be derived from the DFT bin number n according to bpm=n(60/64), where 64 is the number of DFT bins per 1 Hz and 60 is the number of seconds per minute. FIG. 4 also shows the fundamental heart rate HR₀ as a dashed vertical line with a closed arrowhead and the various harmonics HR₁, HR₂, HR₃, HR₄ as dashed vertical lines with open arrowheads. The heart rate fundamental HR₀ shown in FIG. 4 may be determined, for example, using a pulse-oximeter or other type of heart rate monitor and the corresponding hear rate harmonics HR₁, HR₂, HR₃, HR₄ shown in FIG. 4 may be multiples of the heart rate fundamental HR₀.

In some embodiments, obtaining frequency domain data 116 in block 112 involves merely receiving frequency domain data 116 (rather than processing time domain measurement data 114 to obtain frequency domain data 116). In such embodiments, frequency domain data 116 may be received as a part of block 112 from some other source (e.g. from some other measurement or sensing system, from some other processor and/or the like) which may compute frequency domain data 116 and provide same to block 112.

Once frequency domain data 116 is obtained in block 112, method 110 (FIG. 2) proceeds to block 118 which involves using frequency domain data 116 to discern (or estimate the corresponding frequencies of) one or more heart rate harmonics HR₁, HR₂, HR₃, . . . . In some embodiments, block 118 may also involve discerning (or estimating the location of) the heart rate fundamental HR₀. In particular embodiments, block 118 involves discerning a plurality of elements from a set {HR₀, HR₁, HR₂, HR₃, . . . } that includes the heart rate fundamental and the heart rate harmonics. For brevity, these discerned elements from the set of elements {HR₀, HR₁, HR₂, HR₃, . . . }, which are the result or output of block 118, may be referred to herein as discerned harmonic(s) 120 and the set of elements {HR₀, HR₁, HR₂, HR₃, . . . } which includes the heart rate fundamental and the heart rate harmonics may be referred to herein as the heart rate harmonic set, it being noted that unless the context dictates otherwise, discerned harmonic(s) 120 and the heart rate harmonic set may include the heart rate fundamental HR₀.

In the case of the illustrative FIG. 4 frequency domain data 116, it can be seen that frequency domain data 116 exhibits frequency domain peaks at frequency (e.g. DFT bin) locations which correspond to the heart rate fundamental HR₀ and at least a number of the heart rate harmonics HR₁, HR₂, HR₃. Accordingly, in some embodiments, block 118 comprises discerning ascertaining local maxima (peaks) in frequency domain data 116. In some embodiments, such peak detection may comprise application of a suitable power threshold to select peaks of interest (i.e. peaks which may be considered to be candidates for the heart rate harmonic set may be discerned to have local maxima that are greater than the power threshold). An exemplary power threshold 132 that may be used in the block 118 thresholding process is shown as a horizontal line in FIG. 4.

In some embodiments and/or for some particular data, the block 118 thresholding process may be sufficient to discern a plurality of peaks corresponding to members of the heart rate harmonic set (e.g. by suitable selection of a threshold) and discerned harmonics 120 may include such peaks. This is the case, for example, with threshold 132 shown in FIG. 4 which is able to discern a peak corresponding to HR₀ and a peak corresponding to HR₁. In some such embodiments or some such cases, no further processing is performed in block 118 and the data corresponding to these peaks (e.g. their DFT bin and corresponding magnitudes) are output as discerned harmonic(s) 120.

In some embodiments, it may be desirable to set the power threshold of the block 118 thresholding process to be somewhat lower than the exemplary threshold 132 shown in FIG. 4 (e.g. to avoid accidental exclusion of suitable peaks or to discern a greater number of candidate peaks). In some embodiments, the block 118 power threshold is set as some percentile of the cumulative distribution function (CDF) of the amplitudes of frequency domain data 116. For example, the block 118 power threshold may be set to the 75^{th} percentile of the amplitudes of frequency domain data 116. Other suitable CDF percentile thresholds may be used and such percentile thresholds may be configurable (e.g. user-configurable). In other embodiments, other criteria (such as experimental data) may be used to configure the power threshold used to detect peaks in block 118.

An example of a relatively low power threshold 134 that may be used in the block 118 thresholding process is shown as a horizontal line in FIG. 4. Where threshold 134 is applied to frequency domain data 116 as a part of block 118, a number of additional peaks satisfy the block 118 thresholding criteria. Consequently, in some embodiments, it may be desirable to perform one or more additional or alternative evaluation processes (e.g. to evaluate one or more additional or alternative criteria) as a part of the block 118 harmonic-discerning procedure before outputting discerned harmonic(s) 120. Such additional or alternative evaluation processes may help to more accurately discern a plurality of elements from the heart rate harmonic set.

In some embodiments, block 118 may involve an additional or alternative evaluation process of comparing peak-to-peak distances (in the frequency or bin domain) between pairs of peaks (e.g. peaks that satisfy the block 118 thresholding criteria or peaks of frequency domain data generally) and retaining (as potential members of the heart rate harmonic set or discerned harmonic(s) 120) pairs of peaks whose peak-to-peak distances are within a valid heart rate range for the subject under consideration. For example, the two leftmost peaks Pₐ, P_{b} in the FIG. 4 exemplary data have a peak-to-peak separation on the order of 10 bins, which corresponds to about 0.16 Hz or a heart rate of about 0.16(60 sec/min)=9.6 bpm, which, in normal circumstances, is likely be outside the normal heart rate range for a human subject. Consequently, in embodiments which employ this evaluation process, the pair of peaks Pₐ, P_{b} may be rejected as a potential harmonic pair.

In some embodiments, block 118 may involve an additional or alternative evaluation process of comparing the peak-to-peak distances (in the frequency or bin domain) of pairs of peaks to look for a plurality of pairs of peaks that have approximately equal peak-to-peak distances. Pairs of peaks that fit this criteria might be more likely to belong to the heart rate harmonic set. For example, in the in the FIG. 4 exemplary data, peaks Pₐ, P_{c} are separated by approximately 70 bins. Assuming that frequency domain data 116 contains no other pair of peaks that is separated by approximately 70 bins, then the pair of peaks Pₐ, P_{c} may be rejected as being a pair of peaks that belongs to the heart rate harmonic set. Note that although the pair of peaks Pₐ, P_{c} may be rejected in accordance with this criteria, it does not mean that the individual peaks Pₐ, P_{c} are rejected. As another example, consider the pair of peaks P_{b}, P_{c} and the pair of peaks P_{c}, P_{d} in the FIG. 4 exemplary data. Both of these pairs of peaks are separated by about 56 or 57 bins. Since the pair of peaks P_{b}, P_{c} and the pair of peaks P_{c}, P_{d} are separated by approximately equal distances, both of these pairs of peaks may be considered to be candidates for members of the heart rate harmonic set or discerned harmonic(s) 120.

The notion of approximately equal peak-to-peak distances may be set to any suitable threshold. For example, in some embodiments, pairs of peaks may be considered to be approximately equally separated if their separations are less than or equal to a threshold of about 0.1875 Hz (which corresponds to about 12 bins or 11 bpm in the case of the FIG. 4 exemplary data). In other embodiments, this peak-to-peak distance equality threshold may be set to a different value which may be configurable (e.g. user-configurable). This peak-to-peak distance equality threshold can help to account for peak location error which may be due to interference from other spectral components and/or quantization error.

In some embodiments, block 118 may involve an additional or alternative evaluation process of searching for a “path” of peaks, where a path is an ordered set of three or more peaks, whose adjacent members (as determined by the set order) are approximately equidistant (in the frequency or bin domain). The evaluation of pairs of peaks with approximately equal peak-to-peak separation is discussed above. In the case of the FIG. 4 exemplary data, the peaks P_{b}, P_{c}, P_{d} may be considered to be a valid 3-peak path, because the peaks P_{b}, P_{c} have a separation that is approximately equal to the separation of the peaks P_{c}, P_{d}. Similarly, the peaks P_{c}, P_{d}, Pₑ may be considered to be a valid 3-peak path, because the adjacent peaks P_{c}, P_{d} have a separation that is approximately equal to the separation of the adjacent peaks P_{d}, Pₑ and the peaks P_{b}, P_{c}, P_{d}, Pₑ may be considered to be a valid 4-peak path, because the adjacent peaks P_{b}, P_{c} have a separation that is approximately equal to the separation of the adjacent peaks P_{c}, P_{d} and to the separation of the adjacent peaks P_{d}, Pₑ. In contrast, the set of peaks P_{b}, P_{f}, P_{c} may not be considered to be a valid path because the peak-to-peak separation of peaks P_{b}, P_{f} is significantly different from the peak-to-peak separation of peaks P_{f}, P_{c}. In some embodiments, valid paths may be retained as candidates for members of the heart rate harmonic set or discerned harmonic(s) 120 and groups of peaks that do not form valid paths may be rejected.

In some embodiments, the length of paths evaluated in block 118 may be pre-configured (e.g. user configured) at the time of performing block 118. For example, in some embodiments, block 118 may comprise searching for paths of length 3 (i.e. three peaks) or paths of length 4 (i.e. 4 peaks). In other embodiments, block 118 may accommodate paths of different lengths. For example, in some embodiments, block 118 may comprise searching for paths of minimum length 3 and then determining if the lengths of such paths may be increased by evaluating potential adjacent peaks. In such embodiments, any sub-combinations of longer paths may also be retained as paths, provided that they meet the minimum desired path length. For example, a valid path of length 4 may also be retained as two valid paths of length 3 (i.e. the first three elements of the path of length 4 and the last three elements of the path of length 4 are each valid paths of length 3. In some embodiments, the length of paths may be used as a potential selection criteria as between paths.

In some embodiments, block 118 may involve an additional or alternative evaluation process of subjecting any located paths to a further evaluation which may be referred to herein as a harmonic test. For each candidate path, the harmonic test may involve: evaluating the average peak-to-peak separation f̅ of the peaks in the path (in the frequency or bin domain); and evaluating whether the peaks in the path are at (frequency or bin) locations fₚₑₐₖ which are approximately equal to fₚₑₐₖ=i f̅ where i=1, 2, 3 . . . (i.e. a positive integer). This harmonic test comes from the observation that if the peaks of a path are valid estimates of the elements of the heart rate harmonic set, then they should be located at frequencies that are multiples of the fundamental heart rate frequency, whether or not the heart rate fundamental is one of the estimated elements of the heart rate harmonic set. In some embodiments, a peak location fₚₑₐₖ may be considered to be approximately equal to fₚₑₐₖ=i f̅ where i=1, 2, 3 . . . if the location fₚₑₐₖ is within a suitable threshold window around a location i f̅. In some embodiments, this threshold window may be set at i f̅±α and α may be a configurable (e.g. user configurable) constant or α may be some suitable fraction of the average peak-to-peak separation f̅. In some embodiments, α may be 10% of the average peak-to-peak separation f̅. Candidate paths that do not satisfy the harmonic test may be rejected and candidate paths that do satisfy the harmonic test may be retained as candidates for members of the heart rate harmonic set or discerned harmonic(s) 120.

The result/output of block 118 comprises discerned harmonic(s) 120. In some embodiments, discerned harmonic(s) 120 comprise estimates of a plurality of elements of the heart rate harmonic set {HR₀, HR₁, HR₂, HR₃, . . . }. In some embodiments, discerned harmonic(s) 120 comprise a plurality of individual peaks (e.g. local maxima above a suitable threshold) which may correspond to a corresponding plurality of elements of the heart rate harmonic set. Such peaks may be subject to one or more additional or alternative evaluation criteria. In some embodiments, discerned harmonic(s) 120 comprise one or more paths, each path comprising an ordered set of three or more peaks, whose adjacent members (according to the set order) are approximately equidistant (in the frequency or bin domain). Such paths may be subject to one or more additional or alternative evaluation criteria.

Once discerned harmonic(s) 120 are determined in block 118, method 110 proceeds to block 122 which involves using discerned harmonic(s) 120 determine an estimate 124 of a physiological characteristic (e.g. heart rate estimate 124 in the exemplary embodiment described here). Block 122 may comprise determining heart rate estimate 124 to be an average peak-to-peak distance (in the frequency or bin domain) between a plurality of peaks within discerned harmonic(s) 120. For example, in the case of the exemplary FIG. 4 data and in the case where block 118 comprises application of the threshold 132, discerned harmonic(s) 120 comprise only two peaks P_{b}, P_{c}. In such a case, block 122 may determine that heart rate estimate 124 may be the average distance between peaks P_{b}, P_{c} which is approximately 58 bins (or 58(60/64)=54 bpm. As another example, in the case of the exemplary FIG. 4 data and where discerned harmonic(s) 120 comprise a single path (e.g. the path comprising peaks P_{b}, P_{c}, P_{d}, Pₑ), block 122 may determine that heart rate estimate 124 may be the average of the peak-to-peak distances between peaks {P_{b},P_{c}=58 bins; P_{c},P_{d}=56 bins; P_{d},Pₑ=54 bins}, which works out to 56 bins (or 56(60/64)=53 bpm).

In some embodiments and/or in some circumstances, block 122 may involve selecting a subset of the data from within discerned harmonic(s) 120 to use for the purpose of determining heart rate estimate 124. For example, in some embodiments, discerned harmonic(s) 120 may comprise multiple paths and block 122 may comprise selecting a preferred path from among the paths in discerned harmonic(s) 120 and then using the preferred path for the purpose of determining heart rate estimate 124 (e.g. by calculating the average peak-to-peak separation in the preferred path). In some embodiments, the selection of a preferred path from among the paths in discerned harmonic(s) 120 may be based on a comparison of the power of the peaks in each path. By way of non-limiting example, in some embodiments, the preferred path may comprise the path with the highest average power per peak. In some embodiments, the power of a peak may comprise selecting the power (amplitude) of the local maxima that corresponds to the peak. In some embodiments, the power of a peak may comprise an integral (or sum) of the power of frequency domain data 116 in a region or window around a peak. By way of non-limiting example, the frequency or bin domain width of such regions may be selected based on a number of bins on either side of the local maxima, the locations where the peak in the frequency domain data 116 crosses back below the block 118 threshold and/or the like.

In some embodiments, other suitable techniques may additionally or alternatively be used to select a subset of discerned harmonic(s) 120 to be used in block 122 for determining heart rate estimate 124. Such additional or alternative techniques are described in more detail below.

Heart rate estimate 124 is the output of method 110 (FIG. 2). In some embodiments, method 110 may optionally loop back (via path 111) to block 112 for another iteration. Each iteration (loop) of method 110 may involve generating a corresponding heart rate estimate 124. Such looping may be used, for example, where method 110 is used to monitor the heart rate of a subject over a period of time. In such embodiments, each iteration of method 110 may correspond to a heart rate estimate for a corresponding period of time and subsequent iterations may correspond to subsequent time periods. It is not necessary that method 110 loop. In some embodiments, method 110 determines a single heart rate estimate 124 based on a single iteration of blocks 112, 118 and 122.

Specific embodiments and implementations of particular features of method 110 are now described in more detail.

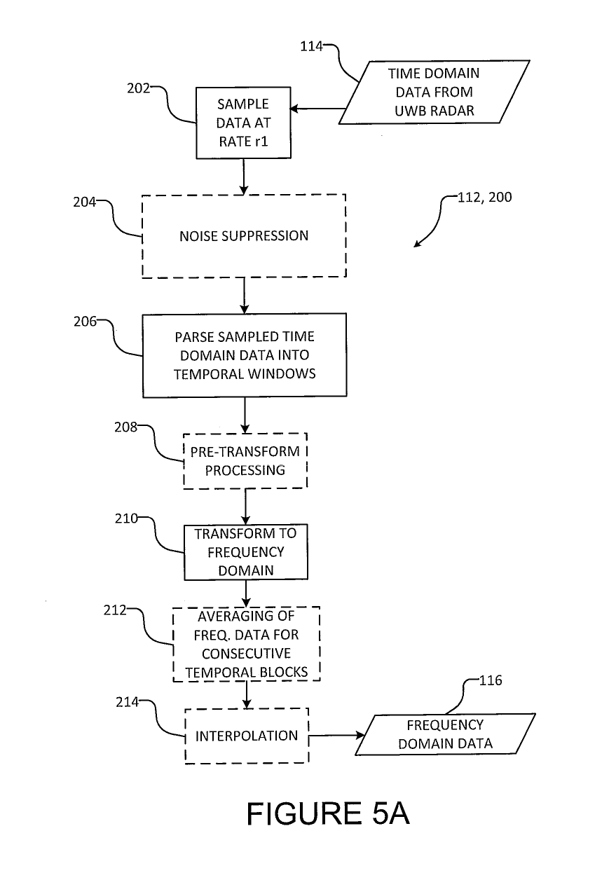

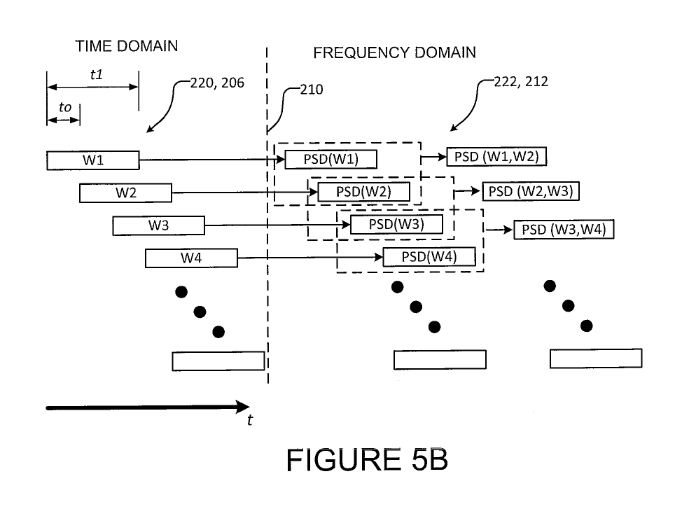

FIG. 5A is a block diagram representation of a method 200 for obtaining frequency domain data 116 according to a particular embodiment. In some embodiments, method 200 of FIG. 5A may be used to implement block 112 of method 110 (FIG. 2). Method 200 may be performed by UWB sensing system 14 (e.g. by digital signal processing system 33 or digital signal processing system 33 in combination with controller 28). In some embodiments, method 200 may be performed in part by UWB radar system 30 under the control of signal processing system 33 and/or controller 28.

Method 200 commences in block 202 which involves receiving time domain data 114 from a UWB radar system (e.g. UWB radar system 30 (FIG. 1A)). As discussed above, time domain data 114 may comprise a version of the radar-received signal received by UWB receiver 30B and down-converted to baseband. As also discussed above, time domain data 114 may be pre-filtered by analog filters (similar to those discussed above in connection with FIG. 3) to separate the heart rate fundamental from the respiration rate fundamental and to block DC. Where method 200 is involved in estimating a heart rate, time domain data 114 may comprise band-pass filtered signal 130 which is passed by heart filter 128 (FIG. 3). In some embodiments, this pre-filtering can be performed as a part of method 200.

Block 202 of the illustrated embodiment comprises sampling time domain data 114 at a first sampling rate r₁. This block 202 first sampling rate r₁ may be relatively low as compared to prior art sampling rates. In some embodiments, this first sampling rate is 128 Hz. In some embodiments, this first sampling rate is less than or equal to 256 Hz. In some embodiments, this sampling rate is less than or equal 1024 Hz.

In block 204, an optional noise suppression process is applied to the data sampled in block 202. In some embodiments, block 204 comprises applying a down-sampling or decimation process to the data sampled in block 202 to generate down-sampled (decimated) time domain data. This block 204 down-sampling process may reduce noise which may be present in the sampled data output from block 202. The optional block 204 down-sampling process may involve taking the average of every j samples of the block 202 data and generating a single sample in the block 204 down-sampled data set for every j samples of the block 202 data. In some embodiments, j=8, although j could be provided with other values. The block 204 down-sampling effectively reduces the block 202 sampling rate to r₂=r₁/j. Thus, in embodiments where r₁=128 Hz and j=8, r₂ is 16 Hz. In some embodiments, the optional noise suppression functionality of block 204 may be provided by different types of operations. By way of non-limiting example, in some embodiments, the block 204 noise suppression functionality could be provided by a moving average filter. Such a moving average filter may not involve a reduction in the effective sampling rate of the block 202 data. In other embodiments, block 204 may comprise applying other suitable averaging or down-sampling filters to the data sampled in block 202 to suppress noise. In some embodiments, block 204 is not necessary.

Method 200 then proceeds to block 206 which involves parsing the sampled time domain data (output from block 202 or from optional block 204) into temporal windows. FIG. 5B is a schematic depiction of a method 220 for parsing time domain measurement data into temporal windows which may be used in block 206 according to a particular embodiment. Method 220 of FIG. 5B involves parsing the sampled time domain data into temporal windows of duration t₁, where each successive window is offset from the preceding window by an offset t₀, where t₀<t₁. For example, in accordance with method 220, a first block 206 temporal window W₁ may include samples of the time domain data output from block 202 or from optional block 204) during the period between t=0 and t=t₁, a second temporal window W₂ includes samples of the time domain data during the period between t=t₀ and t=t₁+t₀, a third temporal window W₃ includes samples of the time domain data during the period between t=2t₀ and t=t₁2t₀, a fourth temporal window W₄ includes samples of the time domain data during the period t=3t₀ and t=t₁+3t₀ and so on.

With this method 220 scheme, each successive temporal window shares samples (of a duration t₁−t₀) with its preceding temporal window. In some embodiments, the ratio of a duration of shared samples between successive windows to a duration of each window (i.e.

$$\frac{{t}_{1} - {t}_{0}}{{t}_{1}} )$$

is in a range of 50% to 90%. In one particular embodiment, this ratio is 75%. In some embodiments, the duration of each window t₁ is selected to be in a range of 5-60 seconds. In one particular embodiment, t₁ is set to be t₁=16 seconds. In some embodiments, the offset t₀ is selected to be in a range of 0.5-30 seconds. In one particular embodiment, t₀ is set to be t₀=4 seconds. For ease of explanation, it will be assumed in the remainder of this description (without loss of generality) that the block 206 windowing is configured such that t₁=16 seconds, t₀=4 seconds and the ratio

$$\frac{{t}_{1} - {t}_{0}}{{t}_{1}}$$

is 75%. Further, it will be assumed without loss of generality that the sampling rate r₁=128 Hz and that above-described block 204 down-sampling occurs with j=8. With these assumptions, each temporal window W₁, W₂, W₃, . . . includes 256 samples.

Returning to FIG. 5A, after parsing the time domain data into finite duration windows in block 206, method 200 proceeds to block 208 which comprises optional pre-transform processing. In some embodiments, the optional block 208 pre-transform processing comprises DC removal and Hamming windowing. Hamming windowing may increase the relative amplitude of the main lobe of spectral components and decrease relative amplitude of the side lobes, which may in turn reduce interference between spectral components. In some embodiments, other types of windowing (e.g. Hanning windowing, Kaiser windowing and/or the like) and/or other types of pre-transform processing may be performed in block 206. This processing may be applied independently to the time domain data in each of the block 206 windows.

Method 200 then proceeds to block 210 which involves transforming the time domain data output from block 208 to the frequency domain. In general, any of the techniques described above in relation to block 112 may be applied independently to each block 206 temporal window W₁, W₂, W₃, . . . of time domain data. In some embodiments, the block 210 transformation involves determining a power spectral density, amplitude spectral density or using some other suitable spectral density estimation technique for each block 206 temporal window W₁, W₂, W₃, . . . of time domain data. For example, in one particular embodiment, block 210 comprises applying a DFT to each block 206 temporal window W₁, W₂, W₃, . . . of time domain data and squaring the magnitude of the result to obtain a MSDFT for each block 206 temporal window. As is the case with block 112 discussed above, any suitable technique may be used to compute the DFT, such as, by way of non-limiting example, any suitable FFT technique. In other embodiments, other suitable spectral transform techniques or other spectral density estimation techniques could be used in block 210 in the place of the MSDFT. Such spectral transform techniques include, by way of non-limiting example, any suitable Fourier Transform technique, any suitable time-frequency transform technique, any suitable transform involving a weighted sum of periodic functions, and/or the like. In some embodiments, it is not necessary to square the amplitude of the transformed data and block 210 may involve using the magnitudes of the transformed data frequency domain data.

In some embodiments, block 210 is the last step of method 200 and results in frequency domain data 116. In the illustrated embodiment, method 200 involves a number of optional procedures shown in blocks 212 and 214. For example, block 212 may reduce noise and block 214 may be used to provide an approximation of a more finely sampled version of the spectrum. The procedures of blocks 212 and 214 are not required. In some embodiments, the procedures of block 212 may be performed without the procedures of block 214 or the procedures of block 214 may be performed without the procedures of block 212. In other embodiments, a more finely sampled version of the spectrum could be obtained by other techniques. For example, such other techniques may involve zero-padding the time domain data from the block 206 temporal windows W_{I}, W₂, W₃, . . . and/or the like. In other embodiments, a higher resolution version of the spectrum could be obtained. For example, a higher resolution version of the spectrum may be obtained by using longer temporal windows W₁, W₂, W₃, . . . (i.e. windows with a larger parameter t₁).

Optional block 212 involves averaging the block 210 frequency domain data for a plurality of successive temporal windows W₁, W₂, W₃, . . . (i.e. windows W₁, W₂, W₃, . . . obtained in block 206). In one particular embodiment, this block 212 averaging is performed for each pair of successive temporal windows W₁, W₂, W₃, . . . . This particular embodiment of block 212 is depicted schematically in the method 222 of FIG. 5B. In the FIG. 5B illustration, the vertical dashed line represents the block 210 transformation of time domain data to frequency domain data. Each block 206 temporal window results in corresponding power spectral density data PSD(W₁), PSD(W₂), PSD(W₃) in the frequency domain. In block 212, successive pairs of frequency domain data are averaged—i.e. PSD(W₁) and PSD(W₂) are averaged to form PSD(W₁,W₂), PSD(W₂) and PSD(W₃) are averaged to form PSD(W₂,W₃), PSD(W₃) and PSD(W₄) are averaged to form PSD(W₃,W₄) and so on. In other embodiments, the spectral densities (frequency domain data) of different numbers of temporal windows could be averaged in block 212.

Method 220 then proceeds to optional block 214 which involves interpolating the averaged spectra from the block 212 averaging process. Any suitable interpolation technique could be used in block 214. In one particular embodiment, block 214 comprises applying a cubic spline interpolation technique. Where the block 212 averaging technique involve the averaging of pairs of successive temporal windows to generate frequency domain data PSD(W₁,W₂), PSD(W₂,W₃), PSD(W₃,W₄) . . . (as in the case in the illustrated embodiment of FIG. 5B), the block 214 interpolation may be applied to these frequency domain data to obtain interpolated and averaged frequency domain data P\tilde{S}D(W1, W2), P\tilde{S}D(W2, W3), P\tilde{S}D(W3, W4) . . . . This interpolated and averaged frequency domain data P\tilde{S}D(W1, W2), P\tilde{S}D(W2, W3), P\tilde{S}D(W3, W4) . . . may be output as frequency domain data 116 shown in FIG. 5A—i.e. the result of method 200 and block 112 (FIG. 2). For brevity but without loss of generality, the remainder of this description, except where otherwise noted or where otherwise discernable from the context, assumes that frequency domain data 116 has the form of interpolated and averaged frequency domain data P\tilde{S}D(W1, W2), P\tilde{S}D(W2, W3), P\tilde{S}D(W3, W4) . . . output from block 214.

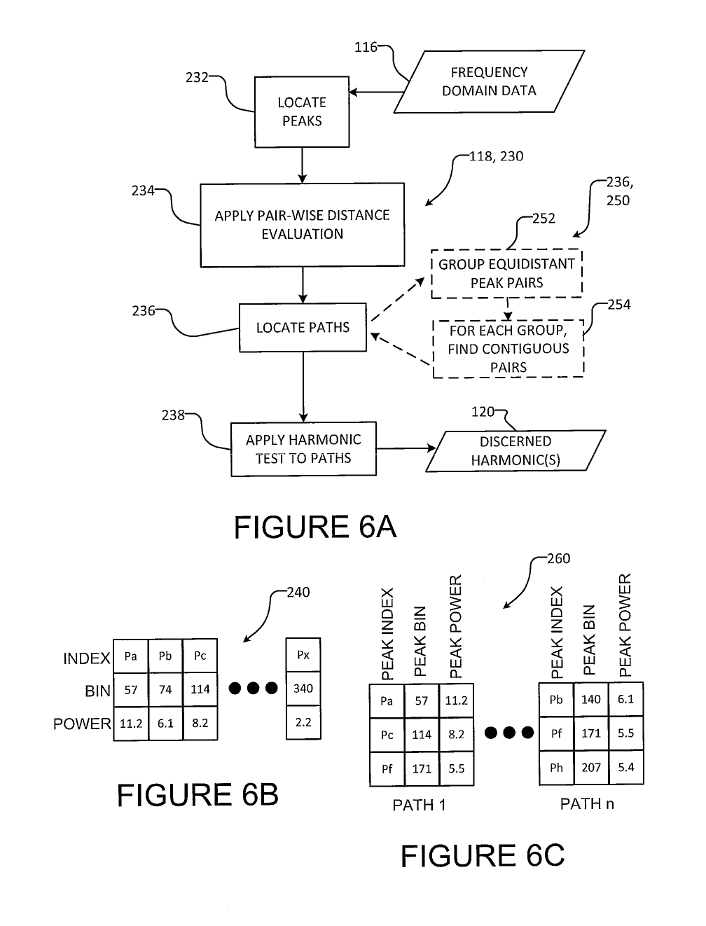

FIG. 6A is a block diagram representation of a method 230 for discerning harmonic(s) and generating corresponding discerned harmonic(s) 120 (e.g. elements of the heart rate harmonic set {HR₀, HR₁, HR₂, HR₃, . . . }) according to a particular embodiment. In some embodiments, method 230 of FIG. 6A may be used to implement block 118 of method 110 (FIG. 2). Method 230 may be performed by UWB sensing system 14 (e.g. by digital signal processing system 33 or digital signal processing system 33 in combination with controller 28). In some embodiments, method 230 may be performed in part by UWB radar system 30 under the control of signal processing system 33 and/or controller 28.

In some embodiments or applications (e.g. when taking a one-time heart rate measurement), method 230 may be performed on a single element of frequency domain data 116 (e.g. P\tilde{S}D(W1, W2)). In some embodiments or applications (e.g. monitoring a subject over a period of time), method 230 may be performed on each element of frequency domain data (once for P\tilde{S}D(W1, W2), once for P\tilde{S}D(W2, W3), once for P\tilde{S}D(W3, W4) . . . ) as such frequency domain data becomes available.

Method 230 commences in block 232 which comprises receiving an element of frequency domain data 116 and locating the local maxima (peaks) in the element of frequency domain data 116. Suitable peak detection procedures are described above in connection with block 118 of method 110 (FIG. 2). Any such peak detection technique may be used in block 232. Block 232 results in a set of peaks (e.g. including their respective bin/frequency domain locations and possibly, for each peak, its corresponding maximum amplitude and/or an indication of its corresponding power (see above discussion of the power associated with an individual peak)). FIG. 6B is a schematic depiction showing a set 240 of peaks which comprises the type of data that may be output from the block 232 peak location process according to a particular embodiment. Each peak in set 240 (Pₐ, P_{b}, . . . Pₓ) includes a corresponding bin/frequency domain location (e.g. bin number) and a corresponding power metric.

Method 230 then proceeds to block 234 which comprises using the set of block 232 to apply a pair-wise distance evaluation to discern pairs of peaks which are spaced apart (in the bin/frequency domain) by distances that correspond to viable heart rates for the particular subject and to reject pairs of peaks which are spaced apart (in the bin/frequency domain) by distances that correspond to heart rates that are not-realistic for the particular subject. Suitable pair-wise distance evaluation techniques are described above in connection with block 118 of method 110 (FIG. 2). Any such pair-wise distance evaluation technique may be used in block 234. The block 234 procedure may be applied to every pair of peaks in the set of peaks generated in block 232. For example, in the case of the exemplary set of peaks 240 shown in FIG. 6B, peak Pₐ may form a pair with each of peaks P_{b}, P_{c}, . . . Pₓ and each of these pairs of peaks may be evaluated using the pair-wise distance evaluation procedures of block 234; similarly peak P_{b} may form a pair with each of peaks P_{c}, P_{d}, . . . Pₓ and each of these pairs of peaks may be evaluated using the pair-wise distance evaluation procedures of block 234; and so on. In some embodiments, pairs of peaks evaluated in block 234 comprise ordered pairs, such that the first peak in each pair has a relatively low frequency/bin location (as compared to the second peak in the pair) and the second peak in each pair has a relatively high frequency/bin location (as compared to the first peak in the pair).

In some embodiments, the pair-wise distance evaluation procedures of block 234 may involve comparing the peak-to-peak distance for each pair of peaks to a lower separation threshold and to an upper separation threshold. Peak pairs who have a peak-to-peak separation that is greater than the upper separation threshold or lower than the lower separation threshold may be rejected as candidate peak pairs. In some embodiments, block 234 may comprise generating a set of candidate peak pairs and then removing candidate peak pairs from this set if they do not satisfy the pair-wise distance evaluation procedures of block 234 (e.g. if their peak-to-peak separation is greater than the upper separation threshold or lower than the lower separation threshold). In some embodiments, these separation thresholds are configurable (e.g. user configurable). In some embodiments, the lower separation threshold is not used and candidate peak pairs are removed from the set of candidate peak pairs only when their peak-to-peak distance is greater than an upper separation threshold. In some embodiments, block 234 is not required and the procedures of block 236 may be applied to every possible pair from within the set of candidate peaks located in block 232.

After applying the pair-wise distance evaluation procedures of block 234, method 230 proceeds to block 236. Block 236 comprises searching the frequency domain data for “paths”. As discussed above, a path is an ordered set of three or more peaks, whose adjacent members (as determined by the set order) are approximately equidistant (in the frequency or bin domain). Suitable techniques for evaluating approximately equal peak-to-peak distances (in the bin/frequency domain) and for selecting paths and member elements of paths are described above in connection with block 118 of method 110 (FIG. 2). Any such technique(s) may be used to search for paths in block 236. In some embodiments, the block 236 path search may be applied to the set of candidate peak pairs resulting from block 234.

FIG. 6A also shows a method 250 which represents one particular optional technique which may be used to search for paths in block 236 according to a particular embodiment. Method 250 starts in block 252 which comprises searching the set of candidate peak pairs for peak pairs which have approximately equal peak-to-peak separation (in the frequency domain) and grouping such approximately equidistant peak pairs into groups, where each group includes only peak pairs whose separations are approximately equal to one another. This block 252 grouping process may be applied to the set of candidate peak pairs resulting from block 234. Any of the above-discussed techniques could be used in block 252 for evaluating approximately equal peak-to-peak distances (in the bin/frequency domain).

Method 250 then proceeds to block 254 which involves, for each of the block 252 groups, locating pluralities of contiguous peak pairs within the group. In this description, two peak pairs may be considered to be contiguous if: the two peak pairs share a common peak; each of the two peak pairs has a non-common peak that is not shared with the other one of the peak pairs; and the common peak is located (in the frequency/bin domain) between the non-common peaks of the two peak pairs. As discussed above, in some embodiments, method 230 may involve creating a list of candidate peak pairs (e.g. in block 234) which are ordered pairs—i.e. where the first peak in each pair has a relatively low frequency/bin location (as compared to the second peak in the pair) and the second peak in each pair has a relatively high frequency/bin location (as compared to the first peak in the pair). In such embodiments, the block 254 search for pluralities of contiguous peak pairs may comprise, for each group of approximately equidistant peak pairs, searching for peak pairs whose second (higher frequency) peak is the same as a first (lower frequency) peak of another peak pair.

It is not necessary, however, that the peak pairs in the candidate set be ordered peak pairs. In embodiments where the peak pairs in the candidate set are not ordered, block 254 may comprise evaluating contiguity in accordance with the a more general approach which comprises, for every two peak pairs within a group of approximately equidistant peak pairs, evaluating each of the three conditions for contiguity. Furthermore, it is not necessary that the path search performed in block 236 be conducted according to the illustrated embodiment of method 250. In other embodiments, paths may be located (from within the set of candidate peak pairs) in accordance with other suitable techniques.

As discussed above in connection with block 118 (FIG. 2), in some embodiments, the lengths of paths located in block 254 (or in block 236 generally) are pre-configured. By way of non-limiting example, in some embodiments, each path comprises a length of three peaks (i.e. two contiguous peak pairs) and, in some embodiments, each path comprises a length of four peaks (i.e. three contiguous peak pairs). Also, as discussed above in connection with block 118 (FIG. 2), in some embodiments, paths located in block 254 (or in block 236 generally) may comprise different lengths.

The result of block 254 (or block 236 generally) is a set of candidate paths. FIG. 6C is a schematic depiction showing a set 260 of paths which comprises the type of data that may be output from the block 236 path detection process according to a particular embodiment. In the particular case of the embodiment illustrated in FIG. 6C, each path in set 260 (PATH_1, PATH_2, . . . PATH_n) includes an ordered set of three peaks and, optionally, for each peak, a corresponding bin/frequency domain location (e.g. bin number) and a corresponding power metric.

After block 236, method 230 proceeds to block 238 which involves application of a harmonic test to the candidate paths that result from the block 236 path search. As discussed above in connection with block 118, the block 238 harmonic test may comprise, for each candidate path: evaluating the average peak-to-peak separation f̅ of the peaks in the path (in the frequency or bin domain); and evaluating whether the peaks in the path are at (frequency or bin) locations fₚₑₐₖ which are approximately equal to fₚₑₐₖ=i f̅ where i=1, 2, 3 . . . . The concept of approximate equality of a peak location fₚₑₐₖ to fₚₑₐₖ=i f̅ where i=1, 2, 3 . . . is also described above. Candidate paths that do not satisfy the block 238 harmonic test may be rejected and removed from the set (e.g. set 260) of candidate paths. Candidate paths that do satisfy the block 238 harmonic test may be retained as candidate paths, whose individual peaks are discerned by method 230 (block 118) to be members of the heart rate harmonic set. Such candidate paths may be output from method 230 (block 118) as discerned harmonic(s) 120. For brevity, but without loss of generality, the remainder of this description, except where otherwise noted or where otherwise discernable from the context, assumes that discerned harmonic(s) 120 comprise one or more candidate paths whose individual peaks are discerned to be members of the heart rate harmonic set.

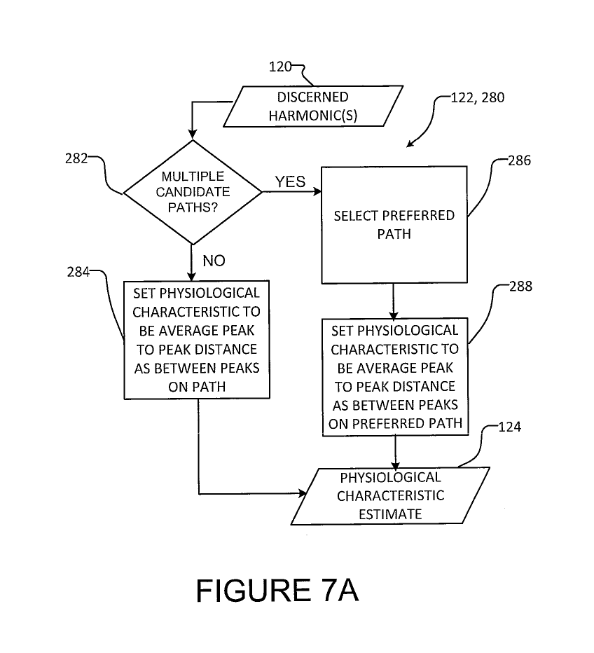

FIG. 7A is a block diagram representation of a method 280 for using discerned harmonic(s) 120 to estimate a physiological characteristic (e.g. heart rate) according to a particular embodiment. In some embodiments, method 280 of FIG. 7A may be used to implement block 122 of method 110 (FIG. 2). Method 280 may be performed by UWB sensing system 14 (e.g. by digital signal processing system 33 or digital signal processing system 33 in combination with controller 28). In some embodiments, method 280 may be performed in part by UWB radar system 30 under the control of signal processing system 33 and/or controller 28.

Method 280 commences in block 282 which involves receiving discerned harmonic(s) 120 (e.g. from block 118 or method 230) and assessing whether discerned harmonic(s) 120 include multiple candidate paths. If the block 282 inquiry is negative (i.e. there is a single path or zero paths in discerned harmonic(s) 120), then method 280 proceeds to block 284. Where there is a single path, block 284 of the illustrated embodiment comprises determining heart rate estimate 124 to correspond to an average peak-to-peak distance (in the frequency/bin domain) between the plurality of peaks in the single path. This process may involve a mapping between the frequency/bin domain to the heart rate domain. In one particular embodiment described herein, the frequency domain data includes 64 bins in 1 Hz and heart rate is usually reported in beats per minute (bpm); consequently mapping from a number n of bins (i.e. the average peak-to-peak distance in the frequency/bin domain) to heart rate (in bpm) might involve a mapping of

$$HR ( bpm ) = n \frac{60}{64} .$$

In other embodiments, this bin/frequency domain to heart rate mapping may be different. Although not expressly shown in FIG. 7A, where there are no paths, block 284 may comprise returning an error indication for heart rate estimate 124.

If the block 282 inquiry is positive and there are multiple paths in discerned harmonic(s), then method 280 proceeds to block 286. Block 286 involves selecting a preferred path from among the multiple paths in discerned harmonics 120. Method 280 then proceeds to block 288 which, in the illustrated embodiment, comprises determining heart rate estimate 124 to correspond to an average peak-to-peak distance (in the frequency/bin domain) between the plurality of peaks in the block 286 preferred path. This block 288 process may be substantially similar to that discussed above for block 284 and may involve a mapping between the bin/frequency domain and the heart rate domain.

A variety of different techniques may be used alone or in combination to select the preferred path in block 286. In some embodiments, a metric based on the amplitudes of the peaks belonging to the paths may be used to select a preferred path in block 286. In some embodiments, the selection metric comprises average peak amplitude of the peaks in the path and the path with the highest average peak amplitude is selected to be the preferred path. In some embodiments, selection of the preferred path in block 286 is based at least in part on the power associated with the peaks in each path. By way of non-limiting example, in some embodiments, the preferred path may comprise the path with the highest average power per peak. Various techniques for determining the power associated with a peak are described above. Any of these techniques may be used in the block 286 selection. Another non-limiting example of a suitable technique which may additionally or alternatively be used, in some embodiments, for the block 286 selection as between paths may comprise selecting the path with the highest aggregate power associated with its peaks to be the preferred path. The power of each peak may be determined as described above and then these peak powers may be aggregated (e.g. summed) as opposed to averaged. This technique might give a preference to relatively long paths, which have a greater number of peaks.

Yet another non-limiting example of a suitable technique which may additionally or alternatively be used, in some embodiments, for the block 286 selection as between paths may comprise a comparison of the peak to non-peak power ratio (PNPR) of the paths and selecting the path with the highest PNPR to be the block 286 preferred path. For each path, determining the PNPR for the path may comprise: determining the power associated with each peak in the path by determining an integral (or sum) of the power in a region or window around the peak; aggregating (e.g. summing) the power associated with each peak in the path; determining an integral (or sum) of the power between peaks to be the integral or sum of non-peak power (i.e. power that is not in the region of a peak); and determining the PNPR to be the ratio of the aggregated peak power to the non-peak power.

Still another non-limiting example of a suitable technique which may additionally or alternatively be used, in some embodiments, for the block 286 selection as between paths may comprise comparing the candidate paths to the paths in a previous iteration of block 286. By way of non-limiting example, some suitable metric (e.g. least squares distance) may be used to compare the locations of peaks in candidate paths to the locations of peaks in a preferred path used in a previous iteration of block 286. The candidate path with the peaks that are closest to those of the previous iteration path may be selected to be the preferred path in the current iteration of block 286.

In other embodiments, other quality metrics could be used to determine a preferred path in block 286.

Method 280 (and block 122) conclude after outputting heart rate estimate 124. As discussed above in relation to method 110 (FIG. 2), at the conclusion of block 122 method 110 may comprise looping back to block 112 via path 111. Each iteration (loop) of method 110 may involve generating a corresponding heart rate estimate 124. In some embodiments, each iteration (loop) of method 110 and corresponding heart rate estimate 124 may be correlated with the parameter t₀ described in FIG. 5B. For example, method 110 may perform one iteration of method 110 and output a corresponding heart rate estimate 124 at a rate corresponding to once for each temporal offset t₀. In such embodiments, each successive iteration of block 112 (e.g. method 200), input time domain data 114 may comprise a new available window W₁, W₂, W₃, . . . of time domain data 114. Similarly, in each successive iteration of block 118 (e.g. method 230), input frequency domain data 116 may comprise a new element P\tilde{S}D(W1, W2), P\tilde{S}D(W2, W3), P\tilde{S}D(W3, W4) . . . of frequency domain data 116.

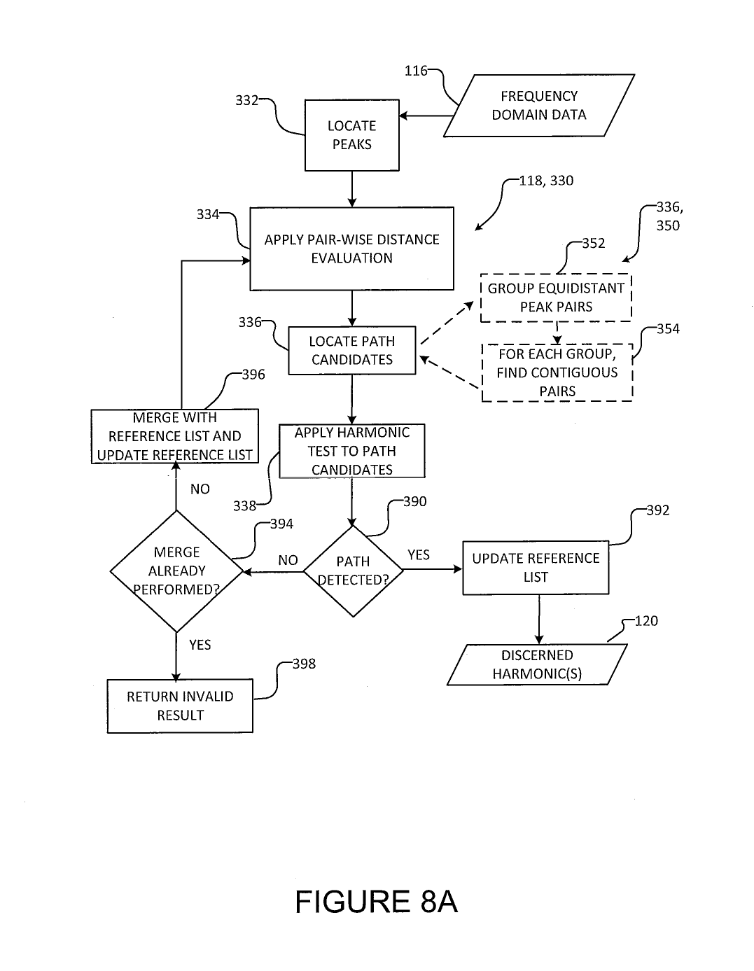

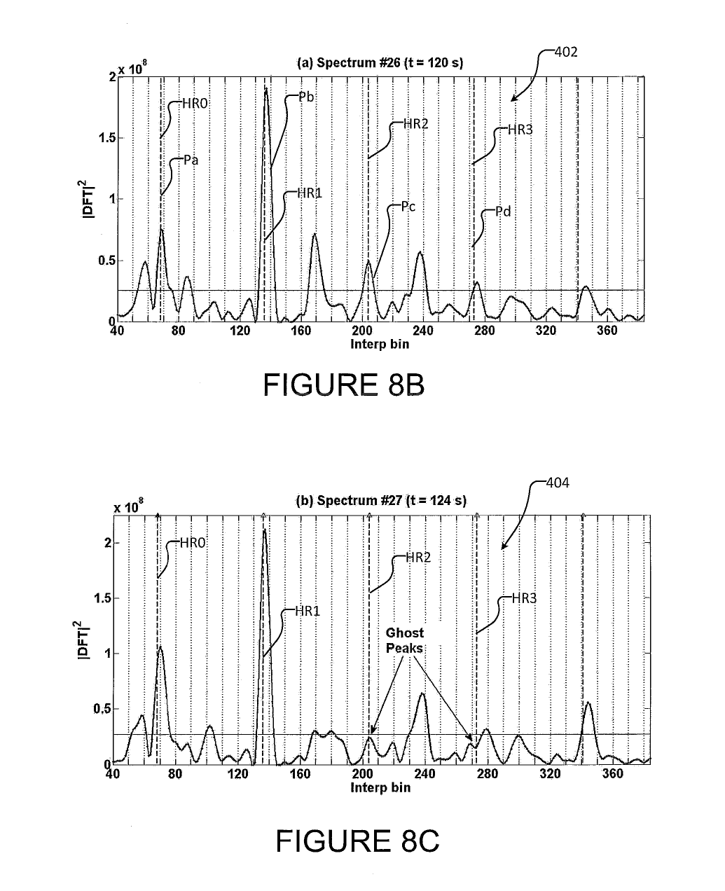

FIG. 8A is a block diagram representation of a method 330 for discerning harmonic(s) and generating corresponding discerned harmonic(s) 120 (e.g. elements of the heart rate harmonic set {HR₀, HR₁, HR₂, HR₃, . . . }) according to another particular embodiment. In some embodiments, method 330 of FIG. 8A may be used to implement block 118 of method 110 (FIG. 2). Method 330 may be performed by UWB sensing system 14 (e.g. by digital signal processing system 33 or digital signal processing system 33 in combination with controller 28). In some embodiments, method 330 may be performed in part by UWB radar system 30 under the control of signal processing system 33 and/or controller 28.

In many respects, method 330 is similar to method 230 (FIG. 6A) and similar references numerals are used to describe blocks of method 330 that are similar to those of method 230, except that the blocks of method 330 are preceded by the numeral “3”, whereas the blocks of method 230 are preceded by the numeral “2”. Method 330 differs from method 230 in that method 330 may take advantage of the looping nature of method 110 (FIG. 2) and may use information from a pervious iteration of method 110 to complement frequency domain data 116 available from the current iteration of method 110. Method 330 takes advantage of the observation that heart rate typically does not change abruptly and successive spectra have peaks at similar frequency/bin locations.

Method 330 commences in block 332 which is substantially similar to block 232 discussed above and involves receiving frequency domain data 116 and locating candidate peaks in frequency domain data. As discussed above, in some embodiments, frequency domain data 116 represents a power spectral density, amplitude spectral density or which corresponds to time domain data 114 from a finite duration time window. As discussed above, in some embodiments, frequency domain data 116 has the form P\tilde{S}D(W1, W2), P\tilde{S}D(W2, W3), P\tilde{S}D(W3, W4) . . . shown in FIG. 5B. Method 330 then proceeds to through block 334, 336, 338 which are substantially similar to blocks 234, 236, 238 described above. The block 336 procedure for locating candidate paths may optional involve performing method 350 (blocks 352, 354), which are substantially similar to method 250 (blocks 252, 254) described above.

Method 330 then proceeds to block 390 which involves an inquiry into whether there are any candidate paths which can be output as discerned harmonic(s) 120. If there are one or more paths, then the block 390 inquiry is positive and method 330 proceeds to block 392. Block 392 involves adding the peaks corresponding to the valid candidate paths (and their corresponding frequency/bin domain data and power data) into a reference list. The information regarding the peaks added to the reference list in block 392 may have the form shown in FIG. 6B. In some embodiments, block 392 may involve determining the preferred path (e.g. in a manner similar to determining the preferred path in block 286 described above) from among the available paths and only adding the peaks corresponding to the preferred path to the reference list. In some embodiments, block 392 may involve adding all of the peaks of all of the available candidate paths to the references list. In some embodiments, block 392 may involve adding some subset of the peaks corresponding to the available candidate paths. In some embodiments, block 392 involves only updating the reference list if the reference list has not already been updated in this iteration of method 330 (e.g. in block 396). At the conclusion of block 392, the available candidate paths (or in some embodiments, the preferred path) are output as discerned harmonic(s) 120.

In some cases, the block 390 inquiry might be negative. That is, there are no valid paths that pass the block 338 harmonic test. This may occur, for example, because heart rate harmonic peaks may be masked by noise, interference and/or the like. If the block 390 inquiry is negative, then method 330 proceeds to block 394 which involves an inquiry into whether the peaks of the current frequency domain data have been merged with the reference list or not. If a merge has not been performed, then method 330 proceeds to block 396 which involves merging the peaks from the reference list with the peaks associated with frequency domain data 116 of the current iteration of method 110 (FIG. 2). For brevity, the peaks associated with frequency domain data 116 of the current iteration of method 110 may be referred to herein as the current peak list. Where a peak on the reference list is spaced apart in the frequency/bin domain (e.g. by a suitable threshold) from the peaks on the current peak list, then the peak from the reference list (and its corresponding data) may be added to the current peak list to provide an updated peak list. However, when a peak on the reference list is within a threshold distance (in the frequency/bin domain) for a peak in the current peak list, then the two peaks (one from the reference list and one from the current peak list) may be merged to provide a single peak on the updated peak list. In some embodiments, block 396 comprises updating the reference list with the updated peak list determined as a part of block 396.

Two peaks may be merged in block 396 according to any suitable technique. In some embodiments, the more powerful of the two peaks is maintained and the other is discarded. In some embodiments, the frequency and power of the two peaks are averaged. In some embodiments, the frequency of the two peaks is averaged, but the power of the more powerful peak is maintained while the power of the less powerful peak is discarded. In some embodiments, other techniques could be used for merging peaks. The threshold distance (in the frequency/bin domain) used in block 396 for determining when peaks should be merged may be configured (e.g. user configured) to any suitable level. In some embodiments, this threshold distance corresponds to 10 bins (which corresponds to about 9 bpm using the above described technique for generating frequency domain data 116). In some embodiments, this threshold distance corresponds to 5 bins (which corresponds to about 4.7 bpm). In some embodiments, other thresholds could be used.

Method 330 then proceeds to block 334 but instead of using the current peak list, method 330 repeats the procedures of blocks 334, 336 and 338 with the updated peak list which includes the current peak list augmented and/or merged with the peaks from the reference list. If the block 390 inquiry is negative when the procedures of blocks 334, 336 and 338 are performed with the updated peak list, then the block 394 inquiry will also be negative and method 330 proceeds to block 398 which involves returning an invalid result for the heart rate estimate. If however, a valid path is detected in block 390, then method 330 proceeds to block 392, where the reference list is updated and one or more paths are output as discerned harmonic(s) 120. In some embodiments, method 330 may not update the reference list in block 392 if the block 390 inquiry is positive based on an updated peak list—i.e. block 392 may only update the reference list when the path(s) that result in the positive block 390 inquiry come from the current peak list. In some embodiments, the reference list may expire if it is not updated within a threshold time or a threshold number of iterations.

FIGS. 8B and 8C show experimental data (in the frequency domain) 402, 404 for successive iterations of method 110. In the particular case of the data shown in FIGS. 8A and 8B, frequency domain data 402 (FIG. 8A) was obtained in accordance with method 200 (FIG. 5A) for a first temporal window and frequency domain data 404 (FIG. 8B) was obtained in accordance with method 200 for a second temporal window t₀=4 seconds later. In the case of frequency domain data 402 shown in FIG. 8B, it can be seen that peaks Pₐ, P_{b}, P_{c}, P_{d} are present at interpolated bins 68, 136, 204 and 273 which in turn correspond to the true heart rate fundamental (HR₀) and the first three heart rate harmonics (HR₁, HR₂, HR₃) respectively. In the case of FIG. 8B, method 230 (FIG. 6A) returns a valid path and the heart rate may be determined in block 122 (e.g. method 280 (FIG. 7A)) to be 64 bpm. Similarly, in the case of FIG. 8B, method 330 detects a path in block 390 and the heart rate may be determined in block 122 (e.g. method 280) to be 64 bpm.

In the case of FIG. 8C, however, peaks corresponding to HR₂ and HR₃ are not present in frequency domain data 404. This may occur because of noise or interference, as discussed above. In the case of FIG. 8C, method 230 would return an invalid result. However, when method 330 is applied to the FIG. 8C data, the reference list (which would include the peaks from the FIG. 8B data) is merged into the FIG. 8C (in block 396) and a valid path can be determined from the merged set of peaks to yield a valid harmonic path having peaks at 69, 137, 208 and 276 (interpolated bins) and a resulting heart rate may be determined in block 122 (e.g. method 280) to be 65 bpm.

Certain implementations of the invention comprise computer processors which execute software instructions which cause the processors to perform a method of the invention. For example, one or more processors in a sensing system or a monitoring system may implement data processing steps in the methods described herein by executing software instructions retrieved from a program memory accessible to the processors. The invention may also be provided in the form of a program product. The program product may comprise any medium which carries a set of computer-readable signals comprising instructions which, when executed by a data processor, cause the data processor to execute a method of the invention. Program products according to the invention may be in any of a wide variety of forms. The program product may comprise, for example, physical (non-transitory) media such as magnetic data storage media including floppy diskettes, hard disk drives, optical data storage media including CD ROMs, DVDs, electronic data storage media including ROMs, flash RAM, or the like. The instructions may be present on the program product in encrypted and/or compressed formats.

Where a component (e.g. a software module, controller, processor, assembly, device, component, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

While a number of exemplary aspects and embodiments are discussed herein, those of skill in the art will recognize certain modifications, permutations, additions and sub-combinations thereof. For example:
In some embodiments, the band-pass filters shown in FIG. 3 may be applied to incoming time domain measurement data 114 as a part of block 112 (see FIG. 2) and may be implemented digitally or in the analog domain.
The description set out above relates in large part to estimating heart rate, but heart rate, is only one example of a physiological characteristic that may be estimated using the systems and methods described herein. In some embodiments, the systems and methods described herein may be used (perhaps with some modifications) to estimate other physiological characteristics, such as respiration rate and/or the like. In some embodiments, the above-described techniques can be used to measure respiration rate even in the presence of subject motion, because subject motion may tend to generate spurious low frequency components that can interfere with the respiration rate fundamental, but which may not interfere as much with the respiration rate harmonics.
In some embodiments, spectrograms may be generated from frequency domain data 116 and multi-target tracking technique(s) may be used to track the spectral components (fundamental and harmonics) over various iterations of method 110, since these spectral components will tend to evolve in time “in formation”.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A method for estimating a physiological characteristic of a subject based on a reflected signal received by a UWB radar system, the method comprising:
processing a signal received from the UWB radar system to obtain a frequency domain representation of the signal;
discerning, from the frequency domain representation, a plurality of elements considered to belong to a physiological characteristic harmonic set {PC₀, PC₁, PC₂, PC₃ . . . }, the physiological characteristic harmonic set comprising a fundamental frequency of the physiological characteristics and harmonic frequencies of the physiological characteristic; and
estimating the physiological characteristic based on the discerned elements of the physiological characteristic harmonic set.

2. A method according to claim 1 wherein processing the signal received from the UWB radar system comprises estimating a spectral density of the signal and the frequency domain representation of the signal comprises the estimated spectral density.

3. A method according to claim 2 wherein processing the signal received from the UWB radar system comprises determining a power spectral density of the signal and the frequency domain representation of the signal comprises the determined power spectral density.

4. A method according to claim 1 wherein discerning the plurality of elements considered to belong to the physiological characteristic harmonic set comprises detecting peaks in the frequency domain representation.

5. A method according to claim 4 wherein detecting peaks comprises locating peaks that are above a threshold, the threshold based on a percentile threshold of a cumulative distribution function of the frequency domain representation.

6. A method according to claim 4 wherein discerning the plurality of elements considered to belong to the physiological characteristic harmonic set comprises searching for paths from among the detected peaks, each path comprising an ordered set of three or more peaks, whose adjacent members, as determined by the set order, are approximately equidistant in the frequency domain; and wherein the plurality of elements considered to belong to the physiological characteristic harmonic set comprise the peaks of one or more candidate paths located from among the detected peaks.

7. A method according to claim 6 wherein searching for paths from among the detected peaks comprises:
considering pairs of peaks from a candidate list of peak pairs;
grouping peak pairs whose peaks are approximately equally spaced apart in the frequency domain into groups of peak pairs; and
for each group of peak pairs, searching the group for contiguous peak pairs, a path comprising two or more contiguous peak pairs.

8. A method according to claim 7 wherein two peak pairs are considered to be contiguous if: the two peak pairs share a common peak; each of the two peak pairs has a non-common peak that is not shared with the other one of the peak pairs; and the common peak is located, in the frequency domain, between the non-common peaks of the two peak pairs.

9. A method according to claim 8 wherein grouping peak pairs whose peaks are approximately equally spaced apart in the frequency domain comprises considering peak pairs to belong to the same group when the peak pairs are within a threshold window of being equally spaced apart in the frequency domain.

10. A method according to claim 9 wherein a size of the threshold window is a configurable parameter.

11. A method according to claim 6 comprising:
subjecting the located paths to a harmonic test, for each path, the harmonic test comprising:
evaluating the average peak-to-peak separation f of the peaks in the path in the frequency domain; and
considering the path to pass the harmonic test if the peaks in the path are at frequency locations fₚₑₐₖ which are approximately equal to fₚₑₐₖ=i f̅ where i is a positive integer; and
keeping only paths that pass the harmonic test in the one or more candidate paths.

12. A method according to claim 7 comprising determining the candidate list of peak pairs based on the detected peaks, determining the candidate list of peak pairs comprising subjecting every possible peak pair from among the detected peaks to a pair-wise distance test, for each peak pair the pair-wise distance test comprising:
determining the peak-to-peak distance, in the frequency domain, between the peaks in the peak pair; and
including only those peak pairs having peak-to-peak distances which are in a range of possible values for the physiological characteristic in the candidate list of peak pairs.

13. A method according to claim 6 wherein if there is only one candidate path located from among the detected peaks, then estimating the physiological characteristic based on the discerned elements of the physiological characteristic harmonic set comprises estimating the physiological characteristic based on the peaks in the one candidate path.

14. A method according to claim 13 wherein the physiological characteristic comprises a heart rate of the subject and estimating the physiological characteristic based on the peaks in the one candidate path comprises estimating the heart rate based on an average peak-to-peak distance, in the frequency domain, between the peaks in the one candidate path.

15. A method according to claim 13 wherein the physiological characteristic comprises a respiration rate of the subject and estimating the physiological characteristic based on the peaks in the one candidate path comprises estimating the respiration rate based on an average peak-to-peak distance, in the frequency domain, between the peaks in the one candidate path.

16. A method according to claim 6 wherein if there are a plurality of candidate paths located from among the detected peaks, then estimating the physiological characteristic based on the discerned elements of the physiological characteristic harmonic set comprises:
selecting a preferred path from among the plurality of candidate paths; and estimating the physiological characteristic based on the peaks in the preferred path.

17. A method according to claim 16 wherein selecting a preferred path from among the plurality of candidate paths comprises selecting the preferred path based at least in part on amplitudes of the peaks in the plurality of candidate paths.

18. A method according to claim 17 wherein selecting the preferred path based at least in part on amplitudes of the peaks in the plurality of candidate paths comprises selecting the preferred path to be the candidate path with a highest average peak amplitude.

19. A method according to claim 17 wherein selecting the preferred path based at least in part on amplitudes of the peaks in the plurality of candidate paths comprises selecting the preferred path to be the candidate path with the highest average power per peak.

20. A method according to claim 17 wherein selecting the preferred path based at least in part on amplitudes of the peaks in the plurality of candidate paths comprises selecting the preferred path to be the candidate path with the highest aggregate power.

21. A method according to claim 17 wherein selecting the preferred path based at least in part on amplitudes of the peaks in the plurality of candidate paths comprises selecting the preferred path to be the candidate path with the highest peak to non-peak power ratio (PNPR).

22. A method according to claim 17 wherein selecting the preferred path based at least in part on amplitudes of the peaks in the plurality of candidate paths comprises selecting the preferred path to be the candidate path that most resembles a path used to determine the physiological characteristic in a previous iteration of the method.

23. A method according to claim 16 wherein the physiological characteristic comprises a heart rate of the subject and estimating the physiological characteristic based on the peaks in the preferred path comprises estimating the heart rate based on an average peak-to-peak distance, in the frequency domain, between the peaks in the preferred path.

24. A method according to claim 16 wherein the physiological characteristic comprises a respiration rate of the subject and estimating the physiological characteristic based on the peaks in the preferred path comprises estimating the respiration rate based on an average peak-to-peak distance, in the frequency domain, between the peaks in the preferred path.

25. A method according to claim 6 wherein if there no candidate paths located from among the detected peaks, then estimating the physiological characteristic based on the discerned elements of the physiological characteristic harmonic set comprises: combining the detected peaks with a reference list of peaks obtained in a previous iteration of the method to thereby provide an updated set of peaks; and searching for paths from among the updated set of peaks; and wherein the plurality of elements considered to belong to the physiological characteristic harmonic set comprise the peaks of one or more candidate paths located from among the updated set of peaks.

26. A method according to claim 25 wherein combining the detected peaks with the reference list of peaks comprises, for each reference peak in the reference list of peaks:
if the reference peak is spaced apart from the detected peaks, adding the reference peak to the updated set of peaks;
if the reference peak is within a threshold distance, in the frequency domain, of a detected peak from among the detected peaks, merging the reference peak and the detected peak to create a merged peak and adding the merged peak to the updated set of peaks.

27. A method according to claim 26 wherein merging the peak and the detected peak comprises creating the merged peak to have a frequency that is the average of the frequencies of the reference peak and the detected peak.

28. A method according to claim 27 wherein merging the peak and the detected peak comprises creating the merged peak to have an amplitude that is one of: an average of the amplitudes of the reference peak and the detected peak; and a higher one of the amplitudes of the reference peak and the detected peak.

29. A system for estimating a physiological characteristic of a subject, the system comprising:
a UWB radar system for directing UWB pulses toward the subject, receiving reflected pulses and generating a signal based on the reflected pulses;
a processor connected to receive the signal from the UWB radar system and configured to:
process the signal to obtain a frequency domain representation of the signal;
discern, from the frequency domain representation, a plurality of elements considered to belong to a physiological characteristic harmonic set {PC₀, PC₁, PC₂, PC₃ . . . }, the physiological characteristic harmonic set comprising a fundamental frequency of the physiological characteristics and harmonic frequencies of the physiological characteristic; and
estimate the physiological characteristic based on the discerned elements of the physiological characteristic harmonic set.

30. A computer program product program product comprising a non-transitory computer-readable medium having executable code configured to cause a processor executing the code to perform a method for estimating a physiological characteristic of a subject based on a reflected signal received by a UWB radar system, the method comprising:
processing a signal received from the UWB radar system to obtain a frequency domain representation of the signal;
discerning, from the frequency domain representation, a plurality of elements considered to belong to a physiological characteristic harmonic set {PC₀, PC₁, PC₂, PC₃ . . . }, the physiological characteristic harmonic set comprising a fundamental frequency of the physiological characteristics and harmonic frequencies of the physiological characteristic; and
estimating the physiological characteristic based on the discerned elements of the physiological characteristic harmonic set.
